SEQUENCE LISTING (2 sequences)

SEQ ID NO: 1 - protein, 25 aa, Homo sapiens
DTHFPICIFCCGCCHRSKCGMCCKT

SEQ ID NO: 2 - protein, 25 aa, Mus musculus
DTNFPICIFCCKCCNNSQCGICCKT

(19) United States
(12) Patent Application Publication
(10) Pub. No.: US 2007/0042460 A1
(43) Pub. Date: Feb. 22, 2007

(54) Oxidation of peptides

(75) Inventors: Jean-Marc Sabatier, Rousset (FR); Ziad Fajloun, Marseille (FR)

Correspondence Address: DARBY & DARBY P.C., P. O. BOX 5257, NEW YORK, NY, 10150-5257, US

(73) Assignee: Cellpep S.A., Paris (FR)

(21) Appl. No.: 10/558,958

(22) PCT Filed: May 28, 2004
(86) PCT No.: PCT/EP04/05953

§ 371 Date: Mar. 13, 2006

(30) Foreign Application Priority Data
May 30, 2003 (GB) 0312352.8

(51) Int. Cl. C12P 21/06; C07K 14/47

(52) U.S. Cl. 435/69.1; 530/324

(57) ABSTRACT

The folding/oxidation of a reduced peptide or partially reduced peptide to form a disulphide bridged peptide is effected by dissolving it in an oxidizing organic solvent, alone or in admixture with water, adding an aqueous alkaline buffer to the solution, and recovering the resultant disulphide bridged peptide. The preferred oxidizing organic solvent is dimethylsulphoxide, which is desirably used as a 10 to 50% aqueous solution. The addition of the aqueous alkaline buffer, which is preferably a 0.2 M Tris-HCI buffer, is preferably added during a period of from 5 to 90 minutes after dissolution of the reduced peptide in the oxidizing organic solvent. The method allows reduced peptides which are insoluble in alkaline conditions to be oxidized and allows reduced peptides which may form stable but inactive oxidized species if treated with dimethylsulphoxide alone to be fully oxidized.

The invention relates to a method for the folding/oxidation of disulphide bridged peptides.

The in vitro folding/oxidation has been extensively analyzed for several proteins as an experimental approach to the in vivo protein folding/oxidation. The differences observed between the folding in vivo and in vitro, such as the time scale of both processes, the involvement of enzymes in native half-cystine pairings in the endoplasmic reticulum of secretory cells, and subcellular interactions of the nascent chain during protein biosynthesis, suggest that this “spontaneous event” is actually a highly complex phenomenon. Nevertheless, although there is no straightforward explanation of the process by which a reduced compound folds/oxidizes to its native structure, there is sufficient evidence from studies in vitro to allow some generalizations: (i) The folding of reduced peptides occurs spontaneously in a given environment (e.g. pH, temperature, and ionic strength), except for some proteolytically activated proteins obtained by processing of their zymogen forms (e.g. α-chymotrypsin, insulin), (ii) the information leading to the stable native structure is mainly determined by the amino acid sequence of the peptide chain through successive short, medium, and long range interatomic interactions, and (iii) peptide folding appears to be a thermodynamically controlled process in which the rate-limiting step is theoretically the formation of the native-like species (lowest Gibbs free energy for the native peptide with respect to all degrees of freedom).

In the solid phase synthesis of a multiple disulphide-bridged polypeptide, one of the most crucial and versatile steps is folding/oxidation of the reduced product. The standard oxidation medium used is generally 0.2 M Tris-HCl or sodium phosphate buffer, pH 8.0-8.5. The kinetics of oxidation, as well as the folding pathway, can be directly monitored by successive analyses of the reaction mixture in analytical C₈/C₁₈ reversed-phase HPLC. Generally, the main peak corresponding to the hydrophobic reduced form of the peptide progressively disappears (at a variable rate) and new peaks corresponding to partially folded/oxidized peptide intermediates are detected. With some exceptions, these unstable intermediates are generally more hydrophilic than is the reduced peptide. The content of the peptide medium can evolve over several days depending on the peptide structure/number of half-cystine residues, but an equilibrium is often reached in less than 40 hours at room temperature. At equilibrium, the oxidation process is completed and a major hydrophilic peak will be observed which corresponds to the fully folded/oxidized target peptide. Total oxidation of the peptide can be verified by monitoring the redox potential with 5,5′ dithiobis(2-nitrobenzoic acid), i.e. Ellman's reagent. The oxidation medium can then be filtered prior to purification since peptide aggregation is frequently observed, presumably associated with intermolecular disulphide bridge formation.

Some particular problems can arise during the folding/oxidation procedure. They include: (i) insolubility of the reduced peptide in usual conditions of oxidation, e.g. neutral or basic pH values resulting in precipitation/aggregation of the peptide, and (ii) formation of stable but inactive oxidized species. The way to solve these problems depends mainly on the individual peptide structure and physicochemical properties, but some chemical additives or modifications of the experimental protocol may help. For example, inclusion in the medium of a redox mixture of 0.1 mM reduced and 1 mM oxidized glutathione accelerates oxidation by thiol-thiol interchange and reshuffling of disulphide bonds, and in some cases enhances the recovery of folded active peptide. The reduced/oxidized glutathione system has been described as acting on the stability of oxidation intermediates as follows: the reduced form stabilizes thiol groups whereas the oxidized form stabilizes half-cystine residues with mixed linkages with glutathione. Thus, disulphide bonds in the intermediates are destabilized by both reduced and oxidized glutathione. Also, it has been reported that guanidine hydrochloride concentration and temperature may influence the solubility of the reduced peptide or oxidation intermediates, and affect the folding pathway. Another method, which has been developed, and applied successfully to the folding/oxidation of insoluble reduced AaH toxin II, is based on a dialysis oxidation system (Sabatier et al., Int. J. Pept. Prot. Res. 30, 125-134 (1987). The reduced molecules are first solubilized in 10% (v/v) acetic acid and then oxidized by air through dialysis against a series of buffers with a slow pH gradient from 2.2 to 8. This procedure is particularly convenient for oxidizing reduced polypeptides that are totally insoluble in neutral or alkaline buffers. Other additives may help peptide oxidation, such as metal ions (e.g. trace amounts of copper), chemical oxidants (e.g. potassium ferricyanide), and natural disulphide interchange enzymes (e.g. thioredoxin, glutaredoxin, protein disulphide isomerase).

U.S. Pat. No. 5,144,006 describes the oxidative folding of peptides using dimethylsulphoxide. Use of a buffer is optional, but there is no description of a buffer being added after dissolution in dimethylsulphoxide. If the optional buffer is used, it is present throughout. We have found that this proposal is not effective in all cases. If the peptide is insoluble in neutral or basic pH values, it will precipitate if one attempts to dissolve it in dimethylsulphoxide and alkaline buffer. However, dimethylsulphoxide alone does not fully oxidize all peptides, and some may form stable but inactive oxidized species.

The invention provides a method for the preparation of a disulphide bridged peptide by oxidation of the equivalent reduced or partially reduced peptide, the method comprising dissolving the reduced peptide or partially reduced in an oxidizing organic solvent, alone or in admixture with water, adding an aqueous alkaline buffer to the solution, and recovering the resultant disulphide bridged peptide.

The reduced or partially reduced peptide can be one produced by chemical synthesis or by a recombinant approach. The preferred oxidizing organic solvent is dimethylsulphoxide, although other oxidizing organic solvents such as diethyl ether may be used instead. Dimethylsulphoxide is preferably used in admixture with water, particularly in mixtures containing from 10 to 50% by volume of dimethylsulphoxide. If the peptide contains tryptophan residues, it is preferred that the dimethylsulphoxide:water mixture should contain not more than 20% by volume of dimethylsulphoxide.

Suitable buffers are saline buffer, sodium phosphate buffer and, especially, 0.2 M Tris-HCl buffer. The pH of the solution should be one which allows oxidation of the peptide, e.g. from 6 to 12, but a range from 8 to 8.5 is preferred.

It is important to add the alkaline buffer after dissolving the peptide or partially reduced peptide in the oxidizing organic solvent, alone or in admixture with water. If the buffer is present when the peptide is dissolved in the oxidizing organic solvent, precipitation may occur. The reduced peptide is preferably left to oxidize in the oxidizing organic solvent for at least 5 minutes and more preferably 10 minutes before adding the alkaline buffer. Addition of the alkaline buffer within a period of approximately 10 to 90 minutes is usually best, although the alkaline buffer can be added later. Addition after more than a day or two is, however, unlikely to produce any greater benefit. If left too long before addition of buffer, stable but inactive oxidized species may form.

We have also found that dilution of peptide solution (<1 mM) does not significantly favour intramolecular half-cystine pairings, in contrast with general belief. The use of a concentrated peptide solution, from 0.5 to 5 mM, facilitates handling and renders easier the task of target peptide purification by preparative C₈/C₁₈ reversed-phase HPLC and/or ion exchange chromatography.

The method of the invention can be carried out on peptides with attached moieties, such as lipopeptides and glycopeptides. It may also be carried out to fold/oxidize unspliced peptides which are subsequently cut to provide the desired peptide.

The method of the invention may be carried out without using any of the additives mentioned above, that is out in the absence of glutathione, guanidine hydrochloride, metal ions, disulphide interchange enzymes and inorganic oxidants.

The invention also provides a peptide oxidation medium comprising an oxidizing organic solvent (e.g. dimethylsulphoxide), water and an aqueous alkaline buffer at a pH of from 6 to 12, preferably from 8 to 8.5.

The invention is illustrated by the following example.

### EXAMPLE

### Application to the Chemical Synthesis of Hepcidin

Amino acid sequence of human hepcidin:
DTHFPICIFCCGCCHRSKCGMCCKT-OH
Amino acid sequence of mouse hepcidin:
DTNFPICIFCCKCCNNSQCGICCKT-OH

The experimental procedure to be used to fold/oxidize a reduced polypeptide, such as hepcidin, is as follows:

Dissolve the crude reduced peptide in an oxidative aqueous/organic solution containing first dimethylsulphoxide/water only (from 10 to 50%, v/v). After ca. 10 min to 1 hour, add a few drops of a buffer at alkaline pH value (e.g. 0.2 M Tris-HCl, pH 8.3). The final peptide concentration could range from 0.5 to 5 mM.

Stir the peptide mixture at room temperature (20-25° C.) for 24 to 150 hours to complete oxidation, then filter (if necessary) and purify the folded/oxidized peptide solution.

The oxidative medium successfully used to fold/oxidize human (25-mer) and mouse (25-mer) hepcidins was dimethylsulphoxide/water/0.2 M Tris-HCl buffer at pH 8.3, at relative solution volumes of 2/2/1.

If the buffer is not added, or is added too late, hepcidin is not obtained because the peptide is incompletely oxidised. If the buffer is present when it is attempted to dissolve the crude reduced peptide is the dimethylsulphoxide/water, precipitation occurs.

1. A method for the preparation of a disulphide bridged peptide by oxidation of a reduced or partially reduced peptide, the method comprising dissolving the reduced peptide or partially reduced peptide in an oxidizing organic solvent, alone or in admixture with water, adding an aqueous alkaline buffer to the solution, and recovering the resultant disulphide bridged peptide.

2. A method according to claim 1 in which the oxidizing organic solvent is dimethylsulphoxide.

3. A method according to claim 2 in which a dimethylsulphoxide : water mixture containing from 10 to 50% by volume of dimethylsulphoxide is used to dissolve the reduced peptide.

4. A method according to claim 1 in which the oxidizing organic solvent is diethyl ether.

5. A method according to claim 1 in which the concentration of the reduced or partially reduced peptide in the solution is from 0.5 to 5 mM.

6. A method according to claim 1 in which the buffer is added during a period of from 5 to 90 minutes after dissolution of the peptide in the oxidizing organic solvent.

7. A method according to claim 1 in which the aqueous alkaline buffer is a saline buffer.

8. A method according to claim 1 in which the aqueous alkaline buffer is 0.2M Tris-HCl buffer.

9. A method according to claim 1 in which the aqueous alkaline buffer is a sodium phosphate buffer.

10. A method according to claim 1 in which the pH of the buffer is from 8.0 to 8.5.

11. A method according to claim 1 for the preparation of hepcidin.

12. A method according to claim 1 for the preparation of human hepcidin.

13. A method according to claim 1 for the preparation of a lipopeptide, a glycopeptide or a peptide having another attached moiety.

14. A method according to claim 1, which method is carried out in the absence of glutathione, guanidine hydrochloride, metal ions, disulphide interchange enzymes or inorganic oxidants.

15. A method according to claim 2 in which the buffer is added during a period of from 5 to 90 minutes after dissolution of the peptide in the oxidizing organic solvent.

16. A method according to claim 4 in which the buffer is added during a period of from 5 to 90 minutes after dissolution of the peptide in the oxidizing organic solvent.

17. A method according to claim 2 in which the concentration of the reduced or partially reduced peptide in the solution is from 0.5 to 5 mM.

18. A method according to claim 4 in which the concentration of the reduced or partially reduced peptide in the solution is from 0.5 to 5 mM.
